SEQUENCE LISTING (78 sequences)

SEQ ID NO: 1 - protein, 31 aa, Homo sapiens
CRVRGGRCAVLSCLPKEEQIGKCSTRGRKCC

SEQ ID NO: 2 - protein, 29 aa, Homo sapiens
CGYGTARCRKKCRSQEYRIGRCPNTYACC

SEQ ID NO: 3 - protein, 30 aa, Homo sapiens
CRRSEGFCQEYCNYMETQVGYCSKKKDACC

SEQ ID NO: 4 - protein, 29 aa, Homo sapiens
CKLGRGKCRKECLENEKPDGNCRLNFLCC

SEQ ID NO: 5 - protein, 31 aa, Homo sapiens
CHMQQGICRLFFCHSGEKKRGICSDPWNRCC

SEQ ID NO: 6 - protein, 29 aa, Homo sapiens
CERPNGSCRDFCLETEIHVGRCLNSRPCC

SEQ ID NO: 7 - protein, 29 aa, Homo sapiens
CNKLKGTCKNNCGKNEELIALCQKSLKCC

SEQ ID NO: 8 - protein, 30 aa, Homo sapiens
CLNLSGVCRRDVCKVVEDQIGACRRRMKCC

SEQ ID NO: 9 - protein, 29 aa, Homo sapiens
CWGKSGRCRTTCKESEVYYILCKTEAKCC

SEQ ID NO: 10 - protein, 29 aa, Homo sapiens
CWNFRGSCRDECLKNERVYVFCVSGKLCC

SEQ ID NO: 11 - protein, 31 aa, Homo sapiens
CWNNYVQGHCRKICRVNEVPEALCENGRYCC

SEQ ID NO: 12 - protein, 29 aa, Homo sapiens
CWNLYGKCRYRCSKKERVYVYCINNKMCC

SEQ ID NO: 13 - protein, 29 aa, Homo sapiens
CWNRSGHCRKQCKDGEAVKDTCKNLRACC

SEQ ID NO: 14 - protein, 28 aa, Homo sapiens
CLMGLGRCRDHCNVDEKEIQKCKMKKCC

SEQ ID NO: 15 - protein, 28 aa, Homo sapiens
CWMDGHCRLLCKDGEDSIIRCRNRKRCC

SEQ ID NO: 16 - protein, 29 aa, Homo sapiens
CMGNSGICRASCKKNEQPYLYCRNCQSCC

SEQ ID NO: 17 - protein, 29 aa, Homo sapiens
CWKGQGACQTYCTRQETYMHLCPDASLCC

SEQ ID NO: 18 - protein, 29 aa, Homo sapiens
CELYQGMCRNACREYEIQYLTCPNDQKCC

SEQ ID NO: 19 - protein, 29 aa, Homo sapiens
CWIIKGHCRKNCKPGEQVKKPCKNGDYCC

SEQ ID NO: 20 - protein, 29 aa, Homo sapiens
CYYGTGRCRKSCKEIERKKEKCGEKHICC

SEQ ID NO: 21 - protein, 30 aa, Homo sapiens
CLGLPKCWNYRCEPLHLAYAFYCLLPTSCC

SEQ ID NO: 22 - protein, 30 aa, Homo sapiens
CVSNTPGYCRTCCHWGETALFMCNASRKCC

SEQ ID NO: 23 - protein, 30 aa, Homo sapiens
CWKNNVGHCRRRCLDTERYILLCRNKLSCC

SEQ ID NO: 24 - protein, 30 aa, Homo sapiens
CFNKVTGYCRKKCKVGERYEIGCLSGKLCC

SEQ ID NO: 25 - protein, 33 aa, Homo sapiens
CLNDVGICKKKCKPEEMHVKNGWAMCGKQRDCC

SEQ ID NO: 26 - protein, 29 aa, Homo sapiens
CWNFRGSCRDECLKNERVYVFCVSGKLCC

SEQ ID NO: 27 - protein, 44 aa, Homo sapiens
IINTLQKYYCRVRGGRCAVLSCLPKEEQIGKCSTRGRKCCRRKK

SEQ ID NO: 28 - protein, 50 aa, Homo sapiens
EFELDRICGYGTARCRKKCRSQEYRIGRCPNTYACCLRKWDESLLNRTKP

SEQ ID NO: 29 - protein, 37 aa, Homo sapiens
LKVVDCRRSEGFCQEYCNYMETQVGYCSKKKDACCLH

SEQ ID NO: 30 - protein, 40 aa, Homo sapiens
EFAVCESCKLGRGKCRKECLENEKPDGNCRLNFLCCRQRI

SEQ ID NO: 31 - protein, 49 aa, Homo sapiens
NTICHMQQGICRLFFCHSGEKKRGICSDPWNRCCVSNTDEEGKEKPEMD

SEQ ID NO: 32 - protein, 47 aa, Homo sapiens
GKFKEICERPNGSCRDFCLETEIHVGRCLNSRPCCLPLGHQPRIEST

SEQ ID NO: 33 - protein, 41 aa, Homo sapiens
NAFFDEKCNKLKGTCKNNCGKNEELIALCQKSLKCCRTIQP

SEQ ID NO: 34 - protein, 44 aa, Homo sapiens
DLGPVEGHCLNLSGVCRRDVCKVVEDQIGACRRRMKCCRTWWIL

SEQ ID NO: 35 - protein, 45 aa, Homo sapiens
EVMKCWGKSGRCRTTCKESEVYYILCKTEAKCCVDPKYVPVKPKL

SEQ ID NO: 36 - protein, 48 aa, Homo sapiens
RIETCWNFRGSCRDECLKNERVYVFCVSGKLCCLKPKDQPHLPQHIKN

SEQ ID NO: 37 - protein, 45 aa, Homo sapiens
TEQLKKCWNNYVQGHCRKICRVNEVPEALCENGRYCCLNIKELEA

SEQ ID NO: 38 - protein, 50 aa, Homo sapiens
TPGGTQRCWNLYGKCRYRCSKKERVYVYCINNKMCCVKPKYQPKERWWPF

SEQ ID NO: 39 - protein, 47 aa, Homo sapiens
PAYSGEKKCWNRSGHCRKQCKDGEAVKDTCKNLRACCIPSNEDHRRV

SEQ ID NO: 40 - protein, 44 aa, Homo sapiens
FIGLRRCLMGLGRCRDHCNVDEKEIQKCKMKKCCVGPKVVKLIK

SEQ ID NO: 41 - protein, 34 aa, Homo sapiens
VECWMDGHCRLLCKDGEDSIIRCRNRKRCCVPSR

SEQ ID NO: 42 - protein, 39 aa, Homo sapiens
HILRCMGNSGICRASCKKNEQPYLYCRNCQSCCLQSYMR

SEQ ID NO: 43 - protein, 39 aa, Homo sapiens
EFKRCWKGQGACQTYCTRQETYMHLCPDASLCCLSYALK

SEQ ID NO: 44 - protein, 39 aa, Homo sapiens
PWNPCELYQGMCRNACREYEIQYLTCPNDQKCCLKLSVK

SEQ ID NO: 45 - protein, 39 aa, Homo sapiens
QKSCWIIKGHCRKNCKPGEQVKKPCKNGDYCCIPSNTDS

SEQ ID NO: 46 - protein, 41 aa, Homo sapiens
GWIRRCYYGTGRCRKSCKEIERKKEKCGEKHICCVPKEKDK

SEQ ID NO: 47 - protein, 35 aa, Homo sapiens
QSSCLGLPKCWNYRCEPLHLAYAFYCLLPTSCCLE

SEQ ID NO: 48 - protein, 41 aa, Homo sapiens
GSKCVSNTPGYCRTCCHWGETALFMCNASRKCCISYSFLPK

SEQ ID NO: 49 - protein, 43 aa, Homo sapiens
FEPQKCWKNNVGHCRRRCLDTERYILLCRNKLSCCISIISHEY

SEQ ID NO: 50 - protein, 40 aa, Homo sapiens
LKKCFNKVTGYCRKKCKVGERYEIGCLSGKLCCANDEEEK

SEQ ID NO: 51 - protein, 43 aa, Homo sapiens
WYVKKCLNDVGICKKKCKPEEMHVKNGWAMCGKQRDCCVPADR

SEQ ID NO: 52 - protein, 34 aa, Homo sapiens
IETCWNFRGSCRDECLKNERVYVFCVSGKLCCLK

SEQ ID NO: 53 - DNA, 201 bp, Homo sapiens
atgaggatccattatcttctgtttgctttgctcttcctgtttttggtgcctgttccaggtcatggaggaatcataaacacattacagaaatattattgcagagtcagaggcggccggtgtgctgtgctcagctgccttccaaaggaggaacagatcggcaagtgctcgacgcgtggccgaaaatgctgccgaagaaagaaa

SEQ ID NO: 54 - DNA, 151 bp, Homo sapiens
cgaatttgaattggacagaatatgtggttatgggactgcccgttgccggaagaaatgtcgcagccaagaatacagaattggaagatgtcccaacacctatgcatgctgtttgagaaaatgggatgagagcttactgaatcgtacaaaaccc

SEQ ID NO: 55 - DNA, 128 bp, Homo sapiens
atttaaaagttgttgactgcaggagaagtgaaggcttctgccaagaatactgtaattatatggaaacacaagtaggctactgctctaaaaagaaagacgcctgctgtttacattaaaactgatgttgc

SEQ ID NO: 56 - DNA, 117 bp, Homo sapiens
tttgctgtctgtgagtcgtgcaagcttggtcggggaaaatgcaggaaggagtgcttggagaatgagaagcccgatggaaattgcaggctgaactttctctgctgcagacagaggatc

SEQ ID NO: 57 - DNA, 179 bp, Homo sapiens
aaataccatctgccgtatgcagcaagggatctgcagactttttttctgccattctggtgagaaaaagcgtgacatttgctctgatccctggaataggtgttgcgtatcaaatacagatgaagaaggaaaagagaaaccagagatggatggcagatctgggatctaaaatataagctccc

SEQ ID NO: 58 - DNA, 197 bp, Homo sapiens
aggggagcgggctactcacctccagccttttgtcatccaggggcaaattcaaggagatctgtgaacgtccaaatggctcctgtcgggacttttgcctcgaaacagaaatccatgttgggagatgtttaaatagccgaccctgctgcctgcctctggggcatcaaccaagaattgagagcactacacccaaaaaggac

SEQ ID NO: 59 - DNA, 214 bp, Homo sapiens
ctcaagacccaccccagtcatgaggactttcccttttctctttgccgtgctcttctttctgaccccagccaagaatgcattttttgatgagaaatgcaacaaacttaaagggacatgcaagaacaattgcgggaaaaatgaagaacttattgctctctgccagaagtctctgaaatgctgtcggaccatccagccatgtgggagcattatagat

SEQ ID NO: 60 - DNA, 197 bp, Homo sapiens
gtgatttgggtcctgtggaaggtcattgtctcaatttgtctggtgtttgcagaagagatgtctgcaaagtagtagaagatcaaattggtgcctgccgaagaaggatgaagtgttgtagaacatggtggattttaatgccaattccaacaccacttatcatgtcagattatcaagaaccccttaaacataagttgaaa

SEQ ID NO: 61 - DNA, 228 bp, Homo sapiens
gaagtcatgaaatgttggggcaagtcaggcaggtgcagaacaacatgtaaagaaagtgaagtatactatatattatgcaaaactgaggctaagtgctgtgtggatcccaagtatgtacctgtaaaaccaaaattaacagacacaaatacaagcctggaatcaacttctgcagtctgacacctctcttccaaccttgagtctcaacatcatgggatcctgcagttctat

SEQ ID NO: 62 - DNA, 146 bp, Homo sapiens
gcaggattgaaacatgttggaattttcgtggctcctgccgtgacgaatgcctgaagaatgaaagggtctatgttttctgcgtgagtggtaaactgtgctgtttgaagcccaaggaccagccacatttaccacagcatataaagaat

SEQ ID NO: 63 - DNA, 367 bp, Homo sapiens
tgaggaaggtagcatagtgtgcagttcactggaccaaaagctttggctgcacctcttctggaaagctggccatggggtcttcatgatcattgcaattctgctgttccagaaacccacagtaaccgaacaacttaagaagtgctggaataactatgtacaaggacattgcaggaaaatctgcagagtaaatgaagtgcctgaggcactatgtgaaaatgggagatactgttgcctcaatatcaaggaactggaagcatgtaaaaaaattacaaagccacctcgtccaaagccagcaacacttgcactgactcttcaagactatgttacaataatagaaaatttcccaagcctgaagacacagtctaca

SEQ ID NO: 64 - DNA, 234 bp, Homo sapiens
ggacttgcagcttcattttgggctgccttagccatgaagctccttttgctgactttgactgtgctgctgctcttatcccagctgactccaggtggcacccaaagatgctggaatctttatggcaaatgccgttacagatgctccaagaaggaaagagtctatgtttactgcataaataataaaatgtgctgcgtgaagcccaagtaccagccaaaagaaaggtggtggccattt

SEQ ID NO: 65 - DNA, 381 bp, Homo sapiens
ttcccaaggaccatgaaactcctgctgctggctcttcctatgcttgtgctcctaccccaagtgatcccagcctatagtggtgaaaaaaaatgctggaacagatcagggcactgcaggaaacaatgcaaagatggagaagcagtgaaagatacatgcaaaaatcttcgagcttgctgcattccatccaatgaagaccacaggcgagttcctgcgacatctcccacacccttgagtgactcaacaccaggaattattgatgatattttaacagtaaggttcacgacagactactttgaagtaagcagcaagaaagatatggttgaagagtctgaggcgggaaggggaactgagacctctcttccaaatgttcaccatagctca

SEQ ID NO: 66 - DNA, 552 bp, Homo sapiens
accatgaagctcctttttcctatctttgccagcctcatgctacagtaccaggtgaacacagaatttattggcttgagacgctgtttaatgggtttggggagatgcagggatcactgcaatgtggatgaaaaagagatacagaaatgcaagatgaaaaaatgttgtgttggaccaaaagtggttaaattgattaaaaactacctacaatatggaacaccaaatgtacttaatgaagacgtccaagaaatgctaaaacctgccaagaattctagtgctgtgatacaaagaaaacatattttatctgttctcccccaaatcaaaagcactagcttttttgctaataccaactttgtcatcattccaaatgccacccctatgaactctgccaccatcagcactatgaccccaggacagatcacatacactgctacttctaccaagagtaacaccaaagaaagcagagattctgccactgcctcgccaccaccagcaccacctccaccaaacatactgccaacaccatcactggagctagaggaagcagaagagcag

SEQ ID NO: 67 - DNA, 209 bp, Homo sapiens
tagagtgttggatggatggacactgccggttgttgtgcaaagatggtgaagacagcatcatacgctgccgaaatcgtaaacggtgctgtgttcctagtcgttatttaacaatccaaccagtaacaattcatggaatccttggctggaccactcctcagatgtccacaacagctccaaaaatgaagacaaatataactaatagatagaaa

SEQ ID NO: 68 - DNA, 338 bp, Homo sapiens
agcaaagctcatctctgccgtgctgcagggaaccctatttccttcccctgcagctcagccacctcctcctctcaggtctgccagccatgaaacttctttacctgtttcttgccatccttctggccatagaagaaccagtgatatcaggcaaacgccacatccttcgatgcatgggtaacagtggaatttgtagggcctcttgcaaaaagaacgaacagccctacctctattgcagaaattgtcagtcctgctgcctccagtcctacatgaggataagcatttctggcaaagaggaaaataccgactggtcttatgagaagcagtggccaagactacct

SEQ ID NO: 69 - DNA, 148 bp, Homo sapiens
tgaattcaaacggtgctggaagggtcaaggggcctgccaaacttactgcacaaggcaagaaacttacatgcacctgtgcccggatgcgtccctgtgctgtctctcctatgcattgaaacctccaccggtccccaagcatgaatatgag

SEQ ID NO: 70 - DNA, 201 bp, Homo sapiens
ccttggaatccatgtgagctttaccaaggcatgtgcagaaacgcctgcagagaatatgaaatccaatacttaacctgcccaaatgatcaaaagtgctgcctgaaactttctgtgaaaataaccagttctaaaaatgtgaaggaggattacgactctaactccaacttgtcagttacaaacagttcaagctactctcacatt

SEQ ID NO: 71 - DNA, 118 bp, Homo sapiens
ccaaaaatcttgctggatcataaaaggacactgcaggaaaaactgcaaacctggtgaacaggttaaaaagccatgtaaaaatggtgactattgctgcattccaagcaacacagattct

SEQ ID NO: 72 - DNA, 170 bp, Homo sapiens
atggatggatcagaaggtgctattatggaactggcagatgcaggaaatcatgcaaagaaattgagaggaagaaagaaaaatgtggggaaaaacatatttgctgtgtccctaaagaaaaggataaactatcacacattcacgaccaaaaagagacaagtgagctatatatc

SEQ ID NO: 73 - DNA, 159 bp, Homo sapiens
caatcctcctgccttggcctcccaaagtgctggaattataggtgtgagccactgcacctggcctatgccttttattgcctcctgcctacctcctgctgtttggaatgtgaaagcaagactggagctctaccttggactatgaaaaacaaggacctcacc

SEQ ID NO: 74 - DNA, 216 bp, Homo sapiens
gggtcaaaatgtgtgagtaacaccccaggatactgcaggacatgttgccactggggggagacagcattgttcatgtgcaacgcttccagaaaatgctgcatcagctactccttcctgccgaagcctgacctaccacagctcatcggtaaccactggcaatcaaggagaagaaacacacaaaggaaagacaagaagcaacaaacgaccgtaacatca

SEQ ID NO: 75 - DNA, 405 bp, Homo sapiens
tttgaaccccaaaaatgttggaagaataatgtaggacattgcagaagacgatgtttagatactgaaaggtacatacttctttgtaggaacaagctatcatgctgcatttctataatatcacatgaatatactcgacgaccagcatttcctgtgattcacctagaggatataacattggattatagtgatgtggactcttttactggttccccagtatctatgttgaatgatctgataacatttgacacaactaaatttggagaaaccatgacacctgagaccaatactcctgagactactatgccaccatctgaggccactactcccgagactactatgccaccatctgagactgctacttccgagactatgccaccaccttctcagacagctcttactcataat

SEQ ID NO: 76 - DNA, 219 bp, Homo sapiens
ctcaaaaaatgcttcaataaagtaacaggctattgcaggaagaaatgcaaggtaggagaaagatatgaaataggatgtctaagtgggaaattatgttgtgctaatgatgaagaagagaaaaaacatgtgtcatttaagaagccacatcaacattctggtgagaagctgagtgtgctgcaggattacatcatcttacccaccatcaccattttcacagtc

SEQ ID NO: 77 - DNA, 333 bp, Homo sapiens
atgaagtccctactgttcacccttgcagtttttatgctcctggcccaattggtctcaggtaattggtatgtgaaaaagtgtctaaacgacgttggaatttgcaagaagaagtgcaaacctgaagagatgcatgtaaagaatggttgggcaatgtgcggcaaacaaagggactgctgtgttccagctgacagacgtgctaattatcctgttttctgtgtccagacaaagactacaagaatttcaacagtaacagcaacaacagcaacaacaactttgatgatgactactgcttcgatgtcttcgatggctcctacccccgtttctcccactggt

SEQ ID NO: 78 - DNA, 141 bp, Homo sapiens
attgaaacatgttggaattttcgtggctcctgccgtgacgaatgcctgaagaatgaaagggtctatgttttctgcgtgagtggtaaactgtgctgtttgaagcccaaggaccagccacatttaccacagcatataaagaat

(19) United States
(12) Patent Application Publication
(10) Pub. No.: US 2009/0124541 A1
(43) Pub. Date: May 14, 2009

(54) Method for the recovery and application of novel human defensins as biologically active proteins for the treatment of infections and other diseases

(75) Inventors: Wolf-Georg Forssmann, Hannover (DE); Knut Adermann, Hannover (DE); Jose-Ramon Conejo-Garcia, Hannover (DE)

Correspondence Address: JACOBSON HOLMAN PLLC, 400 SEVENTH STREET N.W., SUITE 600, WASHINGTON, DC, 20004, US

(73) Assignee: IPF PharmaCeuticals GmbH

(21) Appl. No.: 11/790,770

(22) Filed: Apr. 27, 2007

Related U.S. Application Data
(63) Continuation of application No. 10/332,765, filed on Jan. 13, 2003, now abandoned.

(30) Foreign Application Priority Data
Jul. 11, 2000 (DE) 100 33 505.5

(51) Int. Cl. A61K 38/17; C07K 14/435; A61P 1/00; A61P 31/04; A61P 11/00; C07K 1/00

(52) U.S. Cl. 514/12; 530/324; 530/322; 530/333

(57) ABSTRACT

The invention relates to the novel peptides, derived from human blood, hBD-5 (human beta-defensin-5), hBD-6, hBD-7, hBD-8, hBD-10, hBD-11, hBD-12, hBD-13, hBD-14, hBD-15, hBD-16, hBD-17, hBD-18, hBD-19, hBD-20, hBD-22, hBD-23, hBD-24, hBD-25, hBD-26, hBD-27, hBD-28, hBD-29, hBD-30, hBD-31 and hBD-32 and their derivatives whose structure was elucidated for the purpose of therapeutic, diagnostic and commercial use as medicaments. The peptides can be prepared by biotechnological, recombinant methods, by chemical synthesis as well as by proteolysis from corresponding precursor proteins.

The invention relates to peptides of the human defensin type, a method for recovering such peptides in a pure or partially purified form from human and animal body fluids having the capability of preventing bacterial invasion in inflammatory diseases, nucleic acids coding for such peptides, medicaments containing such peptides, and the use of such peptides for the treatment of various diseases.

These peptides can be recovered, in particular, from hemofiltrate or hemodialysate derived from human and animal blood. These substances have been classified as human defensins and can be used for the purpose of (1) medical and commercial use as a medicament, and (2) analysis of diseases.

The substances having the short names hBD-5 (human beta-defensin-5), hBD-6, hBD-7, hBD-8, hBD-10, hBD-11, hBD-12, hBD-13, hBD-14, hBD-15, hBD-16, hBD-17, hBD-18, hBD-19, hBD-20, hBD-22, hBD-23, hBD-24, hBD-25, hBD-26, hBD-27, hBD-28, hBD-29, hBD-30, hBD-31 and hBD-32 were first obtained from the hemofiltrate of patients suffering from kidney diseases after ultrafiltration with a hemodialysis apparatus and functionally characterized by an antibacterial inhibition test. For the preparation of the defensin peptides, a patented method (Forssmann 1988; DE 3633707 C1) which had previously been invented for the recovery of proteins from hemofiltrate was refined. From the molecules obtained with this method which have a molecular weight of below 20 kD and are filtered off with a veno-venous or arterio-venous shunt connection, the peptide fractions containing the human defensin peptides can be recognized by a function test. The previously known method was used for recovering the raw peptide extracts with which a strong effect was observed upon application of Lehrer's radial diffusion test in that the growth of bacteria in a culture is strongly inhibited under the influence of this substance.

Further, it was established that these biological activities could be concentrated in further purification processes until different homogeneous proteins could finally be identified and their structure elucidated. Advantageously, these substances can be purified from the hemofiltrate which was previously considered worthless, to be used as economically utilizable substances. The peptides according to the invention can be obtained by chemical synthesis and by genetic-engineering production, and they can be employed, inter alia, as a pathognomonic diagnostic symptom for the analysis of inflammatory diseases of the gastrointestinal, respiratory and urogenital tracts as well as other epithelial organs.

The present invention relates to peptides having the following amino acid sequence:

Z_{N}-C-Xₘ-X₁-X-C-X₂-Xₙ-C-X-X-X-X₃-Xₒ-C-Xₚ-C-C-Z_{C}

wherein Z_{N} is an amino acid residue or peptide residue of up to 30 amino acids, Z_{C} is an amino acid residue or peptide residue of up to 30 amino acids;

X=an arbitrary amino acid;

Xₘ=3-6 arbitrary amino acids;

Xₙ=2-3 amino acids;

Xₒ=5-9 amino acids;

Xₚ=4-6 amino acids;

X₁=G, A or P;

X₂=R, K, W Q or A;

X₃ E or H.

Peptides having the following sequences are especially preferred:

| | |
|---|---|
| (a) | hBD-5 |
| | Z_{N2}-CRVRGGRCAVLSCLPKEEQIGKCSTRGRKCC-Z_{C2} |
| (b) | hBD-6 |
| | Z_{N3}-CGYGTARCRKKCRSQEYRIGRCPNTYACC-Z_{C3} |
| (c) | hBD-7 |
| | Z_{N4}-CRRSEGFCQEYCNYMETQVGYCSKKKDACC-Z_{C4} |
| (d) | hBD-8 |
| | Z_{N5}-CKLGRGKCRKECLENEKPDGNCRLNFLCC-Z_{C5} |
| (e) | hBD-10 |
| | Z_{N7}-CHMQQGICRLFFCHSGEKKRGICSDPWNRCC-Z_{C7} |
| (f) | hBD-11 |
| | Z_{N8}-CERPNGSCRDFCLETEIHVGRCLNSRPCC-Z_{C8} |
| (g) | hBD-12 |
| | Z_{N9}-CNKLKGTCKNNCGKNEELIALCQKSLKCC-Z_{C9} |
| (h) | hBD-13 |
| | Z_{N10}-CLNSGVCRRDVCKVVEDQIGACRRRMKCC-Z_{C10} |
| (i) | hBD-14 |
| | Z_{N11}-CWGKSGRCRTTCKESEVYYILCKTEAKCC-Z_{C11} |
| (j) | hBD-15 |
| | Z_{N12}-CWNFRGSCRDECLKNERVYVFCVSGKLCC-Z_{C12} |
| (k) | hBD-16 |
| | Z_{N13}-CWNNYVQGHCRKICRVNEVPEALCENGRYCC-Z_{C13} |
| (l) | hBD-17 |
| | Z_{N14}-CWNLYGKCRYRCSKKERVYVYCINNKMCC-Z_{C14} |
| (m) | hBD-18 |
| | Z_{N15}-CWNRSGHCRKQCKDGEAVKDTCKNLRACC-Z_{C15} |
| (n) | hBD-19 |
| | Z_{N16}-CLMGLGRCRDHCNVDEKEIQKCKMKKCC-Z_{C16} |
| (o) | hBD-20 |
| | Z_{N17}-CWMDGHCRLLCKDGEDSIIRCRNRKRCC-Z_{C17} |
| (p) | Z_{N}Z_{C}hBD-22 |
| | Z_{N19}-CMGNSGICRASCKKNEQPYLYCRNCQSCC-Z_{C19} |
| (q) | hBD-23 |
| | Z_{N20}-CWKGQGACQTYCTRQETYMHLCPDASLCC-Z_{C20} |
| (r) | hBD-24 |
| | Z_{N21}-CELYQGMCRNACREYEIQYLTCPNDQKCC-Z_{C21} |
| (s) | hBD-25 |
| | Z_{N22}-CWIIKGHCRKNCKPGEQVKKPCKNGDYCC-Z_{C22} |
| (t) | hBD-26 |
| | Z_{N23}-CYYGTGRCRKSCKEIERKKEKCGEKHICC-Z_{C23} |
| (u) | hBD-27 |
| | Z_{N24}-CLGLPKCWNYRCEPLHLAYAFYCLLPTSCC-Z_{C24} |
| (v) | hBD-28 |
| | Z_{N25}-CVSNTPGYCRTCCHWGETALFMCNASRKCC-Z_{C25} |
| (w) | hBD-29 |
| | Z_{N26}-CWKNNVGHCRRRCLDTERYILLCRNKLSCC-Z_{C26} |
| (x) | hBD-30 |
| | Z_{N27}-CFNKVTGYCRKKCKVGERYEIGCLSGKLCC-Z_{C27} |
| (y) | hBD-31 |
| | Z_{N28}-CLNDVGICKKKCKPEEMHVKNGWAMCGKQRDCC-Z_{C28} |
| (z) | hBD-32 |
| | Z_{N29}-CWNFRGSCRDECLKNERVYVFCVSGKLCC-Z_{C29} |

wherein

Z_{N2} represents an amino acid residue or peptide residue of up to 30 amino acids, especially the peptide residue IINTLQKYY and its N-terminally truncated fragments;

Z_{C2} represents an amino acid residue or peptide residue of up to 30 amino acids, especially the peptide residue RRKK and its C-terminally truncated fragments;

Z_{N3} represents an amino acid residue or peptide residue of up to 30 amino acids, especially the peptide residue EFELDRI and its N-terminally truncated fragments;

Z_{C3} represents an amino acid residue or peptide residue of up to 30 amino acids, especially the peptide residue LRKWDESLLNRTKP and its C-terminally truncated fragments;

Z_{N4} represents an amino acid residue or peptide residue of up to 30 amino acids, especially the peptide residue LKVVD and its N-terminally truncated fragments;

Z_{C4} represents an amino acid residue or peptide residue of up to 30 amino acids, especially the peptide residue LH;

Z_{N5} represents an amino acid residue or peptide residue of up to 30 amino acids, especially the peptide residue EFAVCES and its N-terminally truncated fragments;

Z_{C5} represents an amino acid residue or peptide residue of up to 30 amino acids, especially the peptide residue RQRI and its C-terminally truncated fragments;

Z_{N7} represents an amino acid residue or peptide residue of up to 30 amino acids, especially the peptide residue NTI and its N-terminally truncated fragments;

Z_{C7} represents an amino acid residue or peptide residue of up to 30 amino acids, especially the peptide residue-VSNTDEEGKEKPEMD and its C terminally truncated fragments;

Z_{N8} represents an amino acid residue or peptide residue of up to 30 amino acids, especially the peptide residue GKFKEI and its N-terminally truncated fragments;

Z_{C8} represents an amino acid residue or peptide residue of up to 30 amino acids, especially the peptide residue LPLGHQPRIEST and its C-terminally truncated fragments;

Z_{N9} represents an amino acid residue or peptide residue of up to 30 amino acids, especially the peptide residue NAFFDEK and its N-terminally truncated fragments;

Z_{C9} represents an amino acid residue or peptide residue of up to 30 amino acids, especially the peptide residue RTIQP and its C-terminally truncated fragments;

Z_{N10} represents an amino acid residue or peptide residue of up to 30 amino acids, especially the peptide residue DLGPVEGH and its N-terminally truncated fragments;

Z_{C10} represents an amino acid residue or peptide residue of up to 30 amino acids, especially the peptide residue RTWWIL and its C-terminally truncated fragments;

Z_{N11} represents an amino acid residue or peptide residue of up to 30 amino acids, especially the peptide residue EVMK and its N-terminally truncated fragments;

Z_{C11} represents an amino acid residue or peptide residue of up to 30 amino acids, especially the peptide residue VDPKYVPVKPKL and its C-terminally truncated fragments;

Z_{N12} represents an amino acid residue or peptide residue of up to 30 amino acids, especially the peptide residue RIET and Its N-terminally truncated fragments;

Z_{C12} represents an amino acid residue or peptide residue of up to 30 amino acids, especially the peptide residue LKPKDQPHLPQHIKN and its C-terminally truncated fragments;

Z_{N13} represents an amino acid residue or peptide residue of up to 30 amino acids, especially the peptide residue TEQLKK and its N-terminally truncated fragments;

Z_{C13} represents an amino acid residue or peptide residue of up to 30 amino acids, especially the peptide residue LNIKELEA and its C-terminally truncated fragments;

Z_{N14} represents an amino acid residue or peptide residue of up to 30 amino acids, especially the peptide residue TPGGTQR and its N-terminally truncated fragments;

Z_{C14} represents an amino acid residue or peptide residue of up to 30 amino acids, especially the peptide residue VKPKYQPKERWWPF and its C-terminally truncated fragments;

Z_{N15} represents an amino acid residue or peptide residue of up to 30 amino acids, especially the peptide residue PAYSGEKK and its N-terminally truncated fragments;

Z_{C15} represents an amino acid residue or peptide residue of up to 30 amino acids, especially the peptide residue IPSNEDHRRV and its C-terminally truncated fragments;

Z_{N16} represents an amino acid residue or peptide residue of up to 30 amino acids, especially the peptide residue FIGLRR and its N-terminally truncated fragments;

Z_{C16} represents an amino acid residue or peptide residue of up to 30 amino acids, especially the peptide residue VGPKVVKLIK and its C-terminally truncated fragments;

Z_{N17} represents an amino acid residue or peptide residue of up to 30 amino acids, especially the peptide residue VE;

Z_{C17} represents an amino acid residue or peptide residue of up to 30 amino acids, especially the peptide residue VPSR and its C-terminally truncated fragments;

Z_{N19} represents an amino acid residue or peptide residue of up to 30 amino acids, especially the peptide residue HILR and its N-terminally truncated fragments;

Z_{C19} represents an amino acid residue or peptide residue of up to 30 amino acids, especially the peptide residue LQSYMR and its C-terminally truncated fragments;

Z_{N20} represents an amino acid residue or peptide residue of up to 30 amino acids, especially the peptide residue EFKR and its N-terminally truncated fragments;

Z_{C20} represents an amino acid residue or peptide residue of up to 30 amino acids, especially the peptide residue LSYALK and its C-terminally truncated fragments;

Z_{N21} represents an amino acid residue or peptide residue of up to 30 amino acids, especially the peptide residue PWNP and its N-terminally truncated fragments;

Z_{C21} represents an amino acid residue or peptide residue of up to 30 amino acids, especially the peptide residue LKLSVK and its C-terminally truncated fragments;

Z_{N22} represents an amino acid residue or peptide residue of up to 30 amino acids, especially the peptide residue QKS and its N-terminally truncated fragments;

Z_{C2} represents an amino acid residue or peptide residue of up to 30 amino acids, especially the peptide residue IPSNTDS and its C-terminally truncated fragments;

Z_{N23} represents an amino acid residue or peptide residue of up to 30 amino acids, especially the peptide residue GWIRR and its N-terminally truncated fragments;

Z_{C23} represents an amino acid residue or peptide residue of up to 30 amino acids, especially the peptide residue VPKEKDK and Its C-terminally truncated fragments;

Z_{N24} represents an amino acid residue or peptide residue of up to 30 amino acids, especially the peptide residue QSS and its N-terminally truncated fragments;

Z_{C24} represents an amino acid residue or peptide residue of up to 30 amino acids, especially the peptide residue LE;

Z_{N25} represents an amino acid residue or peptide residue of up to 30 amino acids, especially the peptide residue GSK and its N-terminally truncated fragments;

Z_{C25} represents an amino acid residue or peptide residue of up to 30 amino acids, especially the peptide residue ISYSFLPK;

Z_{N26} represents an amino acid residue or peptide residue of up to 30 amino acids, especially the peptide residue FEPQK and its N-terminally truncated fragments;

Z_{C26} represents an amino acid residue or peptide residue of up to 30 amino acids, especially the peptide residue ISIISHEY;

Z_{N27} represents an amino acid residue or peptide residue of up to 30 amino acids, especially the peptide residue LKK and its N-terminally truncated fragments;

Z_{C27} represents an amino acid residue or peptide residue of up to 30 amino acids, especially the peptide residue ANDEEEK;

Z_{N28} represents an amino acid residue or peptide residue of up to 30 amino acids, especially the peptide residue WYVKK and its N-terminally truncated fragments;

Z_{C28} represents an amino acid residue or peptide residue of up to 30 amino acids, especially the peptide residue VPADR;

Z_{N29} represents an amino acid residue or peptide residue of up to 30 amino acids, especially the peptide residue IET and its N-terminally truncated fragments;

Z_{C29} represents an amino acid residue or peptide residue of up to 30 amino acids, especially the peptide residue LK;

and their cyclic, amidated, acetylated, sulfated, phosphorylated, glycosylated and oxidized derivatives as well as peptide fragments derived from the above described amino acid sequences.

For the above described novel defensin peptides, the following coding nucleic acid sequences (cDNAs) were found, to which the present invention also relates:

| | |
|---|---|
| (a) | hBD-5 |
| | ATGAGGATCCATTATCTTCTGTTTGCTTTGCTCTTCCTGTTTTTGGT |
| | GCCTGTTCCAGGTCATGGAGGAATCATAAACACATTACAGAAATATT |
| | ATTGCAGAGTCAGAGGCGGCCGGTGTGCTGTGCTCAGCTGCCTTCCA |
| | AAGGAGGAACAGATCGGCAAGTGCTCGACGCGTGGCCGAAAATGCTG |
| | CCGAAGAAAGAAA |
| (b) | hBD-6 |
| | CGAATTTGAATTGGACAGAATATGTGGTTATGGGACTGCCCGTTGCC |
| | GGAAGAAATGTCGCAGCCAAGAATACAGAATTGGAAGATGTCCCAAC |
| | ACCTATGCATGCTGTTTGAGAAAATGGGATGAGAGCTTACTGAATCG |
| | TACAAAACCC |
| (c) | hBD-7 |
| | ATTTAAAAGTTGTTGACTGCAGGAGAAGTGAAGGCTTCTGCCAAGAA |
| | TACTGTAATTATATGGAAACACAAGTAGGCTACTGCTCTAAAAAGAA |
| | AGACGCCTGCTGTTTACATTAAAACTGATGTTGC |
| (d) | hBD-8 |
| | TTGCTGTCTGTGAGTCGTGCAAGCTTGGTCGGGGAAAATGCAGGAAG |
| | GAGTGCTTGGAGAATGAGAAGCCCGATGGAAATTGCAGGCTGAACTT |
| | TCTCTGCTGCAGACAGAGGATC |
| (e) | hBD-10 |
| | AAATACCATCTGCCGTATGCAGCAAGGGATCTGCAGACTTTTTTTCT |
| | GCCATTCTGGTGAGAAAAAGCGTGACATTTGCTCTGATCCCTGGAAT |
| | AGGTGTTGCGTATCAAATACAGATGAAGAAGGAAAAGAGAAACCAGA |
| | GATGGATGGCAGATCTGGGATCTAAAATATAAGCTCCC |
| (f) | hBD-11 |
| | AGGGGAGCGGGCTACTCACCTCCAGCCTTTTGTCATCCAGGGGCAAA |
| | TTCAAGGAGATCTGTGAACGTCCAAATGGCTCCTGTCGGGACTTTTG |
| | CCTCGAAACAGAAATCCATGTTGGGAGATGTTTAAATAGCCGACCCT |
| | GCTGCCTGCCTCTGGGGCATCAACCAAGAATTGAGAGCACTACACCC |
| | AAAAAGGAC |
| (g) | hBD-12 |
| | CTCAAGACCCACCCCAGTCATGAGGACTTTCCCTTTTCTCTTTGCCG |
| | TGCTCTTCTTTCTGACCCCAGCCAAGAATGCATTTTTTGATGAGAAA |
| | TGCAACAAACTTAAAGGGACATGCAAGAACAATTGCGGGAAAAATGA |
| | AGAACTTATTGCTCTCTGCCAGAAGTCTCTGAAATGCTGTCGGACCA |
| | TCCAGCCATGTGGGAGCATTATAGAT |
| (h) | hBD-13 |
| | GTGATTTGGGTCCTGTGGAAGGTCATTGTCTCAATTTGTCTGGTGTT |
| | TGCAGAAGAGATGTCTGCAAAGTAGTAGAAGATCAAATTGGTGCCTG |
| | CCGAAGAAGGATGAAGTGTTGTAGAACATGGTGGATTTTAATGCCAA |
| | TTCCAACACCACTTATCATGTCAGATTATCAAGAACCCCTTAAACAT |
| | AAGTTGAAA |
| (i) | hBD-14 |
| | GAAGTCATGAAATGTTGGGGCAAGTCAGGCAGGTGCAGAACAACATG |
| | TAAAGAAAGTGAAGTATACTATATATTATGCAAAACTGAGGCTAAGT |
| | GCTGTGTGGATCCCAAGTATGTACCTGTAAAACCAAAATTAACAGAC |
| | ACAAATACAAGCCTGGAATCAACTTCTGCAGTCTGACACCTCTCTTC |
| | CAACCTTGAGTCTCAACATCATGGGATCCTGCAGTTCTAT |
| (j) | hBD-15 |
| | GCAGGATTGAAACATGTTGGAATTTTCGTGGCTCCTGCCGTGACGAA |
| | TGCCTGAAGAATGAAAGGGTCTATGTTTTCTGCGTGAGTGGTAAACT |
| | GTGCTGTTTGAAGCCCAAGGACCAGCCACATTTACCACAGCATATAA |
| | AGAAT |
| (k) | hBD-16 |
| | TGAGGAAGGTAGCATAGTGTGCAGTTCACTGGACCAAAAGCTTTGGC |
| | TGCACCTCTTCTGGAAAGCTGGCCATGGGGTCTTCATGATCATTGCA |
| | ATTCTGCTGTTCCAGAAACCCACAGTAACCGAACAACTTAAGAAGTG |
| | CTGGAATAACTATGTACAAGGACATTGCAGGAAAATCTGCAGAGTAA |
| | ATGAAGTGCCTGAGGCACTATGTGAAAATGGGAGATACTGTTGCCTC |
| | AATATCAAGGAACTGGAAGCATGTAAAAAAATTACAAAGCCACCTCG |
| | TCCAAAGCCAGCAACACTTGCACTGACTCTTCAAGACTATGTTACAA |
| | TAATAGAAAATTTCCCAAGCCTGAAGACACAGTCTACA |
| (l) | hBD-17 |
| | GGACTTGCAGCTTCATTTTGGGCTGCCTTAGCCATGAAGCTCCTTTT |
| | GCTGACTTTGACTGTGCTGCTGCTCTTATCCCAGCTGACTCCAGGTG |
| | GCACCCAAAGATGCTGGAATCTTTATGGCAAATGCCGTTACAGATGC |
| | TCCAAGAAGGAAAGAGTCTATGTTTACTGCATAAATAATAAAATGTG |
| | CTGCGTGAAGCCCAAGTACCAGCCAAAAGAAAGGTGGTGGCCATTT |
| (m) | hBD-18 |
| | TTCCCAAGGACCATGAAACTCCTGCTGCTGGCTCTTCCTATGCTTGT |
| | GCTCCTACCCCAAGTGATCCCAGCCTATAGTGGTGAAAAAAAATGCT |
| | GGAACAGATCAGGGCACTGCAGGAAACAATGCAAAGATGGAGAAGCA |
| | GTGAAAGATACATGCAAAAATCTTCGAGCTTGCTGCATTCCATCCAA |
| | TGAAGACCACAGGCGAGTTCCTGCGACATCTCCCACACCCTTGAGTG |
| | ACTCAACACCAGGAATTATTGATGATATTTTAACAGTAAGGTTCACG |
| | ACAGACTACTTTGAAGTAAGCAGCAAGAAAGATATGGTTGAAGAGTC |
| | TGAGGCGGGAAGGGGAACTGAGACCTCTCTTCCAAATGTTCACCATA |
| | GCTCA |
| (n) | hBD19 |
| | ACCATGAAGCTCCTTTTTCCTATCTTTGCCAGCCTCATGCTACAGTA |
| | CCAGGTGAACACAGAATTTATTGGCTTGAGACGCTGTTTAATGGGTT |
| | TGGGGAGATGCAGGGATCACTGCAATGTGGATGAAAAAGAGATACAG |
| | AAATGCAAGATGAAAAAATGTTGTGTTGGACCAAAAGTGGTTAAATT |
| | GATTAAAAACTACCTACAATATGGAACACCAAATGTACTTAATGAAG |
| | ACGTCCAAGAAATGCTAAAACCTGCCAAGAATTCTAGTGCTGTGATA |
| | CAAAGAAAACATATTTTATCTGTTCTCCCCCAAATCAAAAGCACTAG |
| | CTTTTTTGCTAATACCAACTTTGTCATCATTCCAAATGCCACCCCTA |
| | TGAACTCTGCCACCATCAGCACTATGACCCCAGGACAGATCACATAC |
| | ACTGCTACTTCTACCAAGAGTAACACCAAAGAAAGCAGAGATTCTGC |
| | CACTGCCTCGCCACCACCAGCACCACCTCCACCAAACATACTGCCAA |
| | CACCATCACTGGAGCTAGAGGAAGCAGAAGAGCAG |
| (o) | hBD-20 |
| | TAGAGTGTTGGATGGATGGACACTGCCGGTTGTTGTGCAAAGATGGT |
| | GAAGACAGCATCATACGCTGCCGAAATCGTAAACGGTGCTGTGTTCC |
| | TAGTCGTTATTTAACAATCCAACCAGTAACAATTCATGGAATCCTTG |
| | GCTGGACCACTCCTCAGATGTCCACAACAGCTCCAAAAATGAAGACA |
| | AATATAACTAATAGATAGAAA |
| (p) | hBD-22 |
| | AGCAAAGCTCATCTCTGCCGTGCTGCAGGGAACCCTATTTCCTTCCC |
| | CTGCAGCTCAGCCACCTCCTCCTCTCAGGTCTGCCAGCCATGAAACT |
| | TCTTTACCTGTTTCTTGCCATCCTTCTGGCCATAGAAGAACCAGTGA |
| | TATCAGGCAAACGCCACATCCTTCGATGCATGGGTAACAGTGGAATT |
| | TGTAGGGCCTCTTGCAAAAAGAACGAACAGCCCTACCTCTATTGCAG |
| | AAATTGTCAGTCCTGCTGCCTCCAGTCCTACATGAGGATAAGCATTT |
| | CTGGCAAAGAGGAAAATACCGACTGGTCTTATGAGAAGCAGTGGCCA |
| | AGACTACCT |
| (q) | hBD-23 |
| | TGAATTCAAACGGTGCTGGAAGGGTCAAGGGGCCTGCCAAACTTACT |
| | GCACAAGGCAAGAAACTTACATGCACCTGTGCCCGGATGCGTCCCTG |
| | TGCTGTCTCTCCTATGCATTGAAACCTCCACCGGTCCCCAAGCATGA |
| | ATATGAG |
| (r) | hBD-24 |
| | CCTTGGAATCCATGTGAGCTTTACCAAGGCATGTGCAGAAACGCCTG |
| | CAGAGAATATGAAATCCAATACTTAACCTGCCCAAATGATCAAAAGT |
| | GCTGCCTGAAACTTTCTGTGAAAATAACCAGTTCTAAAAATGTGAAG |
| | GAGGATTACGACTCTAACTCCAACTTGTCAGTTACAAACAGTTCAAG |
| | CTACTCTCACATT |
| (s) | hBD-25 |
| | CCAAAAATCTTGCTGGATCATAAAAGGACACTGCAGGAAAAACTGCA |
| | AACCTGGTGAACAGGTTAAAAAGCCATGTAAAAATGGTGACTATTGC |
| | TGCATTCCAAGCAACACAGATTCT |
| (t) | hBD-26 |
| | ATGGATGGATCAGAAGGTGCTATTATGGAACTGGCAGATGCAGGAAA |
| | TCATGCAAAGAAATTGAGAGGAAGAAAGAAAAATGTGGGGAAAAACA |
| | TATTTGCTGTGTCCCTAAAGAAAAGGATAAACTATCACACATTCACG |
| | ACCAAAAAGAGACAAGTGAGCTATATATC |
| (u) | hBD-27 |
| | CAATCCTCCTGCCTTGGCCTCCCAAAGTGCTGGAATTATAGGTGTGA |
| | GCCACTGCACCTGGCCTATGCCTTTTATTGCCTCCTGCCTACCTCCT |
| | GCTGTTTGGAATGTGAAAGCAAGACTGGAGCTCTACCTTGGACTATG |
| | AAAAACAAGGACCTCACC |
| (v) | hBD-28 |
| | GGGTCAAAATGTGTGAGTAACACCCCAGGATACTGCAGGACATGTTG |
| | CCACTGGGGGGAGACAGCATTGTTCATGTGCAACGCTTCCAGAAAAT |
| | GCTGCATCAGCTACTCCTTCCTGCCGAAGCCTGACCTACCACAGCTC |
| | ATCGGTAACCACTGGCAATCAAGGAGAAGAAACACACAAAGGAAAGA |
| | CAAGAAGCAACAAACGACCGTAACATCA |
| (w) | hBD-29 |
| | TTTGAACCCCAAAAATGTTGGAAGAATAATGTAGGACATTGCAGAAG |
| | ACGATGTTTAGATACTGAAAGGTACATACTTCTTTGTAGGAACAAGC |
| | TATCATGCTGCATTTCTATAATATCACATGAATATACTCGACGACCA |
| | GCATTTCCTGTGATTCACCTAGAGGATATAACATTGGATTATAGTGA |
| | TGTGGACTCTTTTACTGGTTCCCCAGTATCTATGTTGAATGATCTGA |
| | TAACATTTGACACAACTAAATTTGGAGAAACCATGACACCTGAGACC |
| | AATACTCCTGAGACTACTATGCCACCATCTGAGGCCACTACTCCCGA |
| | GACTACTATGCCACCATCTGAGACTGCTACTTCCGAGACTATGCCAC |
| | CACCTTCTCAGACAGCTCTTACTCATAAT |
| (x) | hBD-30 |
| | CTCAAAAAATGCTTCAATAAAGTAACAGGCTATTGCAGGAAGAAATG |
| | CAAGGTAGGAGAAAGATATGAAATAGGATGTCTAAGTGGGAAATTAT |
| | GTTGTGCTAATGATGAAGAAGAGAAAAAACATGTGTCATTTAAGAAG |
| | CCACATCAACATTCTGGTGAGAAGCTGAGTGTGCTGCAGGATTACAT |
| | CATCTTACCCACCATCACCATTTTCACAGTC |
| (y) | hBD-31 |
| | ATGAAGTCCCTACTGTTCACCCTTGCAGTTTTTATGCTCCTGGCCCA |
| | ATTGGTCTCAGGTAATTGGTATGTGAAAAAGTGTCTAAACGACGTTG |
| | GAATTTGCAAGAAGAAGTGCAAACCTGAAGAGATGCATGTAAAGAAT |
| | GGTTGGGCAATGTGCGGCAAACAAAGGGACTGCTGTGTTCCAGCTGA |
| | CAGACGTGCTAATTATCCTGTTTTCTGTGTCCAGACAAAGACTACAA |
| | GAATTTCAACAGTAACAGCAACAACAGCAACAACAACTTTGATGATG |
| | ACTACTGCTTCGATGTCTTCGATGGCTCCTACCCCCGTTTCTCCCAC |
| | TGGT |
| (z) | hBD-32 |
| | ATTGAAACATGTTGGAATTTTCGTGGCTCCTGCCGTGACGAATGCCT |
| | GAAGAATGAAAGGGTCTATGTTTTCTGCGTGAGTGGTAAACTGTGCT |
| | GTTTGAAGCCCAAGGACCAGCCACATTTACCACAGCATATAAAGAAT |

While the genes of the novel defensin peptides hBD-5, hBD-6, hBD-7, hBD-8, hBD-10, hBD-11, hBD-12 and hBD-13 were found on chromosome 8 by analyzing the corresponding coding nucleotide sequences, the genes of the novel defensin peptides hBD-14, hBD-15, hBD-16, hBD-17, hBD-18, hBD-19, hBD-20, hBD-22, hBD-23, hBD-24, hBD-25, hBD-26, hBD-27, hBD-28, hBD-29, hBD-30, hBD-31 and hBD-32 according to the invention surprisingly could be assigned to chromosome 20.

Thus, it is a further object of the present invention to provide the novel peptides hBD-5 to hBD-32, which are characterized in that they can be respectively used as a readily obtainable medicament having the biological and therapeutic activity of a natural substance.

The present invention further provides a preparation method for the peptides according to the invention, and the use of the peptides according to the invention as medicaments for various therapeutic and diagnostic indications. For this purpose, the defensin peptides can be used as highly pure substances or, if sufficient for a particular use, within a partially purified peptide mixture, or as a mixture of several of the highly pure defensin peptides according to the invention.

The peptides according to the invention can be employed for the treatment of diseases arising from the bacterial colonization of organs.

The peptides according to the invention can further be employed for the treatment of diseases of the human organism, especially those involving the gastrointestinal tract, the respiratory paths and the urogenital apparatus.

In another embodiment of the invention, the peptides according to the invention can be employed for the treatment of diseases of the human organism, especially those involving the integument and its appendage glands.

The peptides according to the invention can also be employed for the treatment of systemic diseases when there is an overproduction of or deficiency in the defensin peptides, especially by antibodies formed against the defensin peptides, or for use in substitution therapy.

In another embodiment of the invention, the peptides according to the invention can be employed for the treatment of chronic diseases which are in part associated with the diseases already mentioned by using them in an appropriate form for the treatment.

The peptides according to the invention can further be employed for the treatment of diseases in an acute stage.

The peptides according to the invention can be employed for the treatment of fertility disorders, especially in diseases involving oocyte-related spermatic penetration disorders and implantation disorders as well as maturation disorders in the male reproduction apparatus, and as a contraceptive.

The peptides according to the invention can be employed for the diagnosis of the diseases already mentioned, for example, by preparing antibodies against one or more of the peptides according to the invention or their derivatives or fragments and measuring the blood concentration of one or more of the peptides according to the invention by immunological methods.

The present invention further relates to various methods for preparing the novel defensin peptides according to the invention or their derivatives, characterized in that they are prepared by prokaryotic or eukaryotic expression and purified by chromatography, and to another method for preparing the defensin peptides or their derivatives by isolating them from human blood by chromatographic methods in a known manner, and finally to a method for preparing the defensin peptides or their derivatives by preparing these defensin peptides by the usual methods of solid-phase and liquid-phase synthesis from the protected amino acids which are contained in the stated sequence, deblocking and purifying it with the usual chromatographic methods.

The defensin peptides are chemically synthesized and formulated as medicaments. The preparation by genetic engineering using usual vectors has also been established. On this route, the novel defensin peptides are prepared in both (1) prokaryotic and (2) eukaryotic organisms. For this purpose, various expression vectors are available on a routine basis for secretory or direct cytoplasmic expression.

The medicinal formulations contain one or more of the novel defensin peptides according to the invention or a physiologically acceptable salt of such peptides. The form and composition of the medicaments which contain one or more of the novel defensin peptides depends on the route of administration. The medicaments containing one or more of the novel defensin peptides can be administered parenterally, intranasally, orally and by inhalation. Preferably, these medicaments containing one or more of the novel defensin peptides are packaged with an injection preparation either as a solution or as a lyophilizate for dissolution immediately before use. The medicinal formulations may also contain auxiliary agents which are required for filling, contribute to the solubility, stability or sterility of the medicament or increase the efficiency of uptake into the body.

The daily dose to be administered of the defensin peptides according to the invention depends on the indication and the use of particular derivatives. For i.v./i.m. injection, it is within a range of from 100 to 1200 units (μg)/day, and for daily subcutaneous injection, it is preferably from 300 to 2400 units (μg)/day.

The determination of the biological activity for the novel defensin peptides according to the invention is based on measurements against internationally used reference preparations for antibiotic substances.

The novel defensin peptides hBD-5, hBD-6, hBD-7, hBD-8, hBD-10, hBD-11, hBD-12, hBD-13, hBD-14, hBD-15, hBD-16, hBD-17, hBD-18, hBD-19, hBD-20, hBD-22, hBD-23, hBD-24, hBD-25, hBD-26, hBD-27, hBD-28, hBD-29, hBD-30, hBD-31 and hBD-32 according to the invention are characterized by also being suitable, in particular, for the long-term therapy of infectious diseases, because they have an excellent biological effectiveness and, on the other hand, do not trigger an immune response even in permanent treatment.

Due to the biological activity of the defensin peptides according to the invention, it is shown that the preparations according to the Invention may be further employed as agents for the therapy of infectious diseases of many epithelial organs.

For determining the activity, the peptides hBD10, hBD17 and hBD19 were tested illustratively for their antimicrobial effects. In a radial diffusion assay, the activities stated in Table 1 could be measured for the peptides against different bacterial strains. In the Table, (+) means the formation of an inhibition halo, and (−) means no formation of an inhibition halo.

TABLE 1

| | hBD10 | hBD17 | hBD19 |
|---|---|---|---|
| Escherichia coli | (+) | (+) | (+) |
| Staphylococcus carnosus | (+) | (+) | (+) |
| Saccharomyces cerevisiae | (+) | (+) | (−) |

For a more precise determination of the antibiotic activity, the minimum inhibitory concentration (MIC) of the above mentioned defensins was determined by standard methods. The results are stated in Table 2, the MIC values corresponding to concentrations in [μg/ml] (nd=not measured).

TABLE 2

| | hBD10 | hBD17 | hBD19 |
|---|---|---|---|
| Escherichia coli | nd | nd | nd |
| Staphylococcus carnosus | <50 | <25 | <25 |
| Saccharomyces cerevisiae | nd | nd | nd |

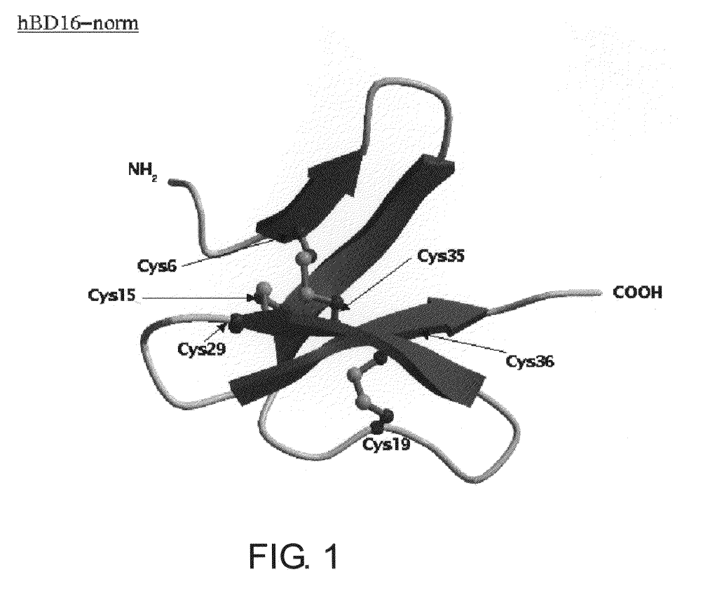

Further, structural analyses were performed with hBD16. FIG. 1 shows the NMR structure of hBD16 found in solution.

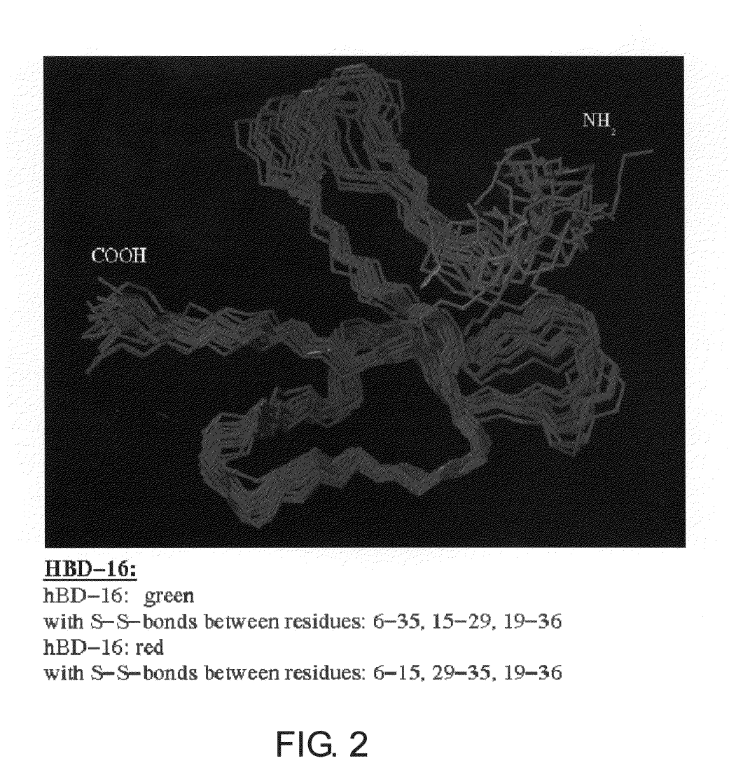

The spatial position of the cysteines Cys 6, 15, 29 and 35 shows that the bridging of these positions not necessarily means a structural change which results in a reduction in activity. This could be shown by the comparison of two bridging patterns (FIG. 2).

1-69. (canceled)

70: An isolated peptide consisting of the following amino acid sequence:
Z_{N}-C-Xₘ-X₁-X-C-X₂-Xₙ-C-X-X-X-X₃-Xₒ-C-Xₚ-C-C-Z_{C}
wherein Z_{N} is an amino acid residue or peptide residue of up to 10 amino acids, Z_{C} is an amino acid residue or peptide residue of up to 10 amino acids;
X=an arbitrary amino acid;
Xₘ=3-6 arbitrary amino acids;
Xₙ=2-3 amino acids;
Xₒ=5-9 amino acids;
Xₚ=4-6 amino acids;
X₁=G, A or P;
X₂=R, K, W, Q or A;
X₃=E or H.

71: The peptide according to claim 70 consisting of the amino acid sequence (bb) hBD-6
Z_{N3}-CGYGTARCRKKCRSQEYRIGRCPNTYACC-Z_{C3}
wherein Z_{N3} represents an amino acid residue or peptide residue of up to 10 amino acids, especially the peptide residue EFELDRI and its—terminally truncated fragments, and Z_{C3} represents an amino acid residue or peptide residue of up to 10 amino acids, especially the peptide residue LRKWDESLLNRTKP and its C-terminally truncated fragments.

72: The peptide according to claim 70, wherein said peptides are the cyclic, amidated, acetylated, sulfated, phosphorylated, glycosylated and oxidized derivatives as well as peptide fragments derived from the above described amino acid sequences and having a similar biological activity.

73: A method for preparing the defensin peptides or their derivatives and fragments according to claim 70, characterized in that they are prepared by prokaryotic or eukaryotic expression and purified.

74: The method according to claim 73, characterized in that the defensin peptides or their derivatives are prepared by the usual methods of chemical solid-phase and liquid-phase peptide synthesis from the protected amino acids which are contained in the stated sequences, deblocked and purified by per se known methods

75. A medicament containing one or more of the defensin peptides or their derivatives or fragments according to claim 70 as an active ingredient in addition to usual auxiliary agents and additives.

76: A method of using one or more of the defensin peptides or their derivatives or fragments according to claim 70 comprising administering the peptide, derivative, or fragment to a person in need thereof for the treatment of diseases of the human organism, especially those involving the gastrointestinal tract, the respiratory paths and the urogenital apparatus.

77: A method of using one or more of the defensin peptides or their derivatives or fragments according to claim 70 comprising administering the peptide, derivative, or fragment to a person in need thereof for the treatment of diseases of the human organism, especially those involving the integument and its appendage glands.

78: A method of using one or more of the defensin peptides or their derivatives or fragments according to claim 70 comprising administering the peptide, derivative, or fragment to a person in need thereof for the treatment of systemic diseases when there is an overproduction of or deficiency in the defensin peptides, especially by antibodies formed against the defensin peptides, or for substitution therapy.

79: A method of using one or more of the defensin peptides or their derivatives or fragments according to claim 70 for the treatment of diseases arising from the bacterial colonization of organs comprising administering the peptide, derivative, or fragment to a person in need thereof for the treatment of said diseases that are chronic diseases.

80: A method of using one or more of the defensin peptides or their derivatives or fragments according to claim 70 for the treatment of diseases arising from the bacterial colonization of organs comprising administering the peptide, derivative, or fragment to a person in need thereof for the treatment of said diseases that are acute by using them in an appropriate form for the treatment in the intensive care of such diseases.

81: A method of using one or more of the defensin peptides or their derivatives or fragments according to claim 70 comprising administering the peptide, derivative, or fragment to a person in need thereof for the treatment of fertility disorders, especially in diseases involving oocyte-related spermatic penetration disorders and implantation disorders as well as maturation disorders in the male reproduction apparatus, and as a contraceptive.

82: A method of using one or more of the defensin peptides or their derivatives or fragments according to claim 70 comprising administering the peptide, derivative, or fragment to a person in need thereof for the diagnosis of diseases, by preparing specific antibodies against one or more of the defensin peptides or their derivatives or fragments and measuring the blood concentration of one or more of the defensin peptides by immunological methods.

83: A method of using one or more of the defensin peptides or their derivatives or fragments according to claim 70 comprising administering the peptide, derivative, or fragment to a person in need thereof in different galenic dosage forms, especially in a lyophilized form taken up with mannitol in sterile ampules for dissolution in physiological saline and/or infusion solutions for repeated singular injection and/or permanent infusion in amounts of from 300 micrograms to 300 milligrams of one or more of the defensin peptides per therapy unit.

84: A method of using one or more of the defensin peptides or their derivatives or fragments according to claim 70 comprising administering the peptide, derivative, or fragment to a person in need thereof in different galenic dosage forms, especially in a lyophilized form taken up with mannitol in sterile ampules for dissolution in physiological saline and/or infusion solutions for repeated singular injection and/or permanent infusion in amounts of from 300 micrograms to 300 milligrams of one or more of the defensin peptides per therapy unit.
